(19) United States
(12) Patent Application Publication
(10) Pub. No.: US 2011/0021507 A1
(43) Pub. Date: Jan. 27, 2011

(54) INHIBITING ANTIPSYCHOTIC-INDUCED WEIGHT GAIN

(75) Inventors: Brian Seed, Boston, MA (US); Jordan Mechanic, Sunnyvale, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Theracos, Inc., Sunnyvale, CA (US)

(21) Appl. No.: 12/835,395

(22) Filed: Jul. 13, 2010

Related U.S. Application Data
(60) Provisional application No. 61/227,588, filed on Jul. 22, 2009.

(51) Int. Cl. A61K 31/551; A61P 25/18; A61P 3/04

(52) U.S. Cl. 514/220

(57) ABSTRACT

The present invention provides methods, compositions and kits for preventing, inhibiting, reducing and reversing weight gain induced by antipsychotic medications, for example olanzapine and clozapine, by co-administration of an M₁R-selective antagonist, for example, telenzepine.

### CROSS-REFERENCE TO RELATED APPLICATIONS

The present application claims the benefit of U.S. Provisional Application No. 61/227,588, filed on Jul. 22, 2010, the disclosure of which is hereby incorporated by reference in its entirety for all purposes.

### FIELD OF THE INVENTION

The present invention provides methods, compositions and kits for preventing, inhibiting, reducing and/or reversing the undesirable side effect of weight gain associated with administration of an antipsychotic selected from olanzapine and clozapine by co-administration of a selective muscarinic receptor M₁ antagonist, for example, telenzepine.

### BACKGROUND OF THE INVENTION

Excessive bodyweight gain is an adverse side effect of treatment with atypical antipsychotic drugs, including olanzapine and clozapine. Among the different atypical antipsychotics, undesirable bodyweight gain is commonly experienced in patients taking olanzapine or clozapine. In fact, olanzapine and clozapine are known to produce the greatest bodyweight gain. As many as 30% of patients being administered olanzapine may experience bodyweight gain of greater than 7%. Numerous pharmacological adjunctive treatments, including co-administration with nizatidine, amantadine, reboxetine, topiramate, sibutramine and metformin, have been tried to counteract olanzapine-induced weight gain with inconclusive or contradictory results. See, e.g., Baptista, et al., CNS Drugs (2008) 22(6): 477-495. There remains a need for better strategies to counteract undesirable weight gain associated with administration of antipsychotic drugs such as olanzapine and clozapine.

### BRIEF SUMMARY OF THE INVENTION

The present invention provides methods of preventing, inhibiting, reducing or reversing weight gain in a subject receiving treatment with an antipsychotic selected from olanzapine and clozapine, comprising co-administering to the subject therapeutically effective amounts of the antipsychotic and an M₁R-selective antagonist.

In some embodiments, the antipsychotic and the M₁R-selective antagonist are concurrently administered. In some embodiments, the antipsychotic and the M₁R-selective antagonist are sequentially administered.

In some embodiments, the subject is human.

In some embodiments, the delivery of at least one of the antipsychotic and the M₁R-selective antagonist is sustained release.

In some embodiments, the M₁R-selective antagonist is selected from the group consisting of telenzepine and pirenzepine. In some embodiments, the M₁R-selective antagonist is telenzepine.

In some embodiments, the antipsychotic is olanzapine. In some embodiments, the antipsychotic is clozapine.

In some embodiments, an antidepressant is co-administered. In some embodiments, lithium is co-administered. In some embodiments, valproate is co-administered.

In a further aspect, the invention provides compositions comprising a mixture of therapeutically effective amounts of an antipsychotic selected from olanzapine and clozapine and an M₁R-selective antagonist.

In some embodiments, the mixture is formulated for sustained release delivery of at least one of the antipsychotic and the M₁R-selective antagonist.

In some embodiments, the M₁R-selective antagonist is selected from the group consisting of telenzepine and pirenzepine. In some embodiments, the M₁R-selective antagonist is telenzepine.

In some embodiments, the antipsychotic is olanzapine. In some embodiments, the antipsychotic is clozapine.

In a related aspect, the methods provide kits comprising a combination of therapeutically effective amounts of an antipsychotic selected from olanzapine and clozapine and an M1R-selective antagonist.

In some embodiments, the antipsychotic and the M₁R-selective antagonist are formulated in a mixture. In some embodiments, at least one of the antipsychotic and the M₁R-selective antagonist are formulated for sustained release delivery.

In some embodiments, the M₁R-selective antagonist is selected from the group consisting of telenzepine and pirenzepine. In some embodiments, the M₁R-selective antagonist is telenzepine.

In some embodiments, the antipsychotic is olanzapine. In some embodiments, the antipsychotic is clozapine.

In some embodiments, the kits further comprise an antidepressant. In some embodiments, the kits further comprise lithium. In some embodiments, the kits further comprise valproate.

Further embodiments of the methods, compositions and kits are as described herein.

### DEFINITIONS

The term “obese” or “obesity” refers to an individual who has a body mass index (BMI) of 30 kg/m² or more. Obesity also can be defined on the basis of body fat content: greater than 25% body fat content for a male or more than 30% body fat content for a female. A “morbidly obese” individual has a body mass index greater than 35 kg/m².

The term “overweight” refers to an individual who has a body mass index of 25 kg/m² or more, but less than 30 kg/m².

The term “body mass index” or “BMI” refers to a weight to height ratio measurement that estimates whether an individual's weight is appropriate for their height. As used herein, an individual's body mass index is calculated as follows:

BMI=(pounds×700)/(height in inches)

Or

BMI=(kilograms)/(height in meters)

The term “baseline body weight” refers to the body weight presented by the individual at the initiation of treatment.

As used herein, “administering” means oral (“po”) administration, administration as a suppository, topical contact, intravenous (“iv”), intraperitoneal (“ip”), intramuscular (“im”), intralesional, intranasal or subcutaneous (“sc”) administration, or the implantation of a slow-release device e.g., a mini-osmotic pump or erodible implant, to a subject. Administration is by any route including parenteral and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The terms “systemic administration” and “systemically administered” refer to a method of administering a compound or composition to a mammal so that the compound or composition is delivered to sites in the body, including the targeted site of pharmaceutical action, via the circulatory system. Systemic administration includes, but is not limited to, oral, intranasal, rectal and parenteral (i.e., other than through the alimentary tract, such as intramuscular, intravenous, intra-arterial, transdermal and subcutaneous) administration, with the proviso that, as used herein, systemic administration does not include direct administration to the brain region by means other than via the circulatory system, such as intrathecal injection and intracranial administration.

The term “co-administer” refers to the simultaneous presence of two active agents in the blood of an individual. Active agents that are co-administered can be concurrently or sequentially delivered.

As used herein, the terms “treating” and “treatment” refer to delaying the onset of, retarding or reversing the progress of, or alleviating or preventing either the disease or condition to which the term applies, or one or more symptoms of such disease or condition.

The terms “patient,” “subject” or “individual” interchangeably refers to a mammal, for example, a human or a non-human mammal, including primates (e.g., macaque, pan troglodyte, pongo), a domesticated mammal (e.g., felines, canines), an agricultural mammal (e.g., bovine, ovine, porcine, equine) and a laboratory mammal or rodent (e.g., rattus, murine, lagomorpha, hamster, guinea pig).

The phrase “antipsychotic-induced weight gain” refers to the side effect of weight gain experienced by patients receiving a therapeutic regimen of an antipsychotic, for example olanzapine and clozapine.

As used herein, the terms “selective muscarinic receptor M₁ antagonist” and “M₁R-selective antagonist” refer to a muscarinic acetylcholine receptor antagonist that exhibits preferential interaction with the muscarinic receptor M₁ subtype in comparison to the muscarinic receptor subtypes M₂ and M₃. Exemplified M₁R-selective antagonists include, but are not limited to, pirenzepine and telenzepine. Preferential binding need not be complete. For example, despite comparable affinities for M₁ and M₄ receptor subtypes, pirenzepine is classified as an M₁R-selective antagonist.

Preferential binding of a M₁R-selective antagonist can be measured in a competitive displacement assay. A M₁R-selective antagonist will preferentially displace a known KR-selective ligand (e.g., pirenzepine and/or telenzepine) in comparison to known M₂ (e.g., tripitramine, himbacine, methoctramine) and M₃ (e.g., darifenacin, hexahydrosiladiphenidol) selective ligands. Alternatively, a M₁R-selective antagonist will preferentially displace a nonselective muscarinic ligand (e.g., quinuclidinyl benzilate (QNB), N-methylscopolamine (NMS)) from an M₁ receptor subtype in comparison to displacing the non-selective muscarinic ligand from binding to the M₂ and M₃ receptor subtypes. The relative potencies for displacement of radiolabeled competitors can be expressed in terms of the concentration at which 50% of the competitor is displaced (IC₅₀), or in terms of an equilibrium dissociation constant (K_{d}). The IC₅₀ value and/or the equilibrium dissociation constant can be calculated using available software by entering the values of detected labeled ligand in the presence of titrated amounts of unlabeled test compound (e.g., LIGAND (Munson, P. J., and Rodbard, D., Anal. Biochem. (1980) 107:220-39 or DATAPLOT, National Technical Information Services). A M₁R-selective antagonist will have an IC₅₀ value or a K_{d} value for binding to an M₁ receptor subtype that is at least about 3-fold less, preferably at least about 10-fold less, and more preferably at least about 30-fold less than its IC₅₀ value or K_{d} value for binding to M₂ and M₃ receptor subtypes. Applicable radioligand binding assays, using radiolabeled NMS or QNB, are disclosed in Buckley, et al., Molecular Pharmacology (1989) 35:469-76 and Bolden, et al, J Pharmacol Exp Ther. (1992) 260:576-80.

As used herein, the phrase “consisting essentially of” refers to the genera or species of active pharmaceutical agents included in a method or composition, as well as any excipients inactive for the intended purpose of the methods or compositions. In some embodiments, the phrase “consisting essentially of” expressly excludes the inclusion of one or more additional active agents other than a M₁R-selective antagonist and an antipsychotic such as olanzapine or clozapine. In some embodiments, additional active agents that are expressly excluded include one or more of an antidepressant or an antipsychotic agent other than olanzapine or clozapine, a prolactin inhibitor, a prolactin stimulator, a 5-HT receptor antagonist, a 5-HT receptor agonist, a NK-1 receptor antagonist, a cyclo-oxygenase-2 (“COX-2”) inhibitor and/or a dipeptidylpeptidase IV inhibitor.

The terms “controlled release,” “sustained release,” “extended release,” and “timed release” are intended to refer interchangeably to any drug-containing formulation in which release of the drug is not immediate, i.e., with a “controlled release” formulation, oral administration does not result in immediate release of the drug into an absorption pool. The terms are used interchangeably with “nonimmediate release” as defined in Remington: The Science and Practice of Pharmacy, 21^{st} Ed., University of the Sciences in Philadelphia (USIP), Lippincott Williams & Wilkins (2005). As discussed therein, immediate and nonimmediate release can be defined kinetically by reference to the following equation:

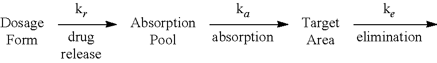

The “absorption pool” represents a solution of the drug administered at a particular absorption site, and kᵣ, kₐ and kₑ are first-order rate constants for (1) release of the drug from the formulation, (2) absorption, and (3) elimination, respectively. For immediate release dosage forms, the rate constant for drug release kᵣ is far greater than the absorption rate constant kₐ. For controlled release formulations, the opposite is true, i.e., kᵣ<<kₐ, such that the rate of release of drug from the dosage form is the rate-limiting step in the delivery of the drug to the target area.

The terms “sustained release” and “extended release” are used in their conventional sense to refer to a drug formulation that provides for gradual release of a drug over an extended period of time, for example, 12 hours or more, and that preferably, although not necessarily, results in substantially steady-state blood levels of a drug over an extended time period.

As used herein, the term “delayed release” refers to a pharmaceutical preparation that passes through the stomach intact and dissolves in the small intestine.

As used herein, “synergy” or “synergistic” interchangeably refer to the combined effects of two active agents that are greater than their additive effects. Synergy can also be achieved by producing an efficacious effect with combined inefficacious doses of two active agents. The measure of synergy is independent of statistical significance.

### BRIEF DESCRIPTION OF THE DRAWINGS

FIG. 1 illustrates the effects of chronic telenzepine administration on olanzapine-induced weight gain in a rat model. Arrow indicates pump implantation and solid bar over the x-axis indicates the drug treatment interval. Telenzepine was administered at a dose of 5 mg/kg/day. Olanzapine was administered at a dose of 10 mg/kg/day. The solid line indicates the effect of OLZ administered alone (OLZ-Induced weight gain), while dashed lines indicate groups receiving TZP, either alone (dashed line) or co-administered with the 10.0 mg/kg OLZ dose (dot-dash-dot line). The dotted line indicates the weight gain in animals receiving only Vehicle treatments.

### DETAILED DESCRIPTION

### 1. Introduction

The present invention is based, in part, on the unexpected and surprising discovery that a selective muscarinic receptor M₁ antagonist, for example, telenzepine, is useful in counteracting the undesirable side effect of weight gain associated with administration of antipsychotic drugs such as olanzapine and clozapine. It is unexpected that a selective muscarinic receptor M₁ antagonist, for example, telenzepine, has sufficient potency in promoting weight loss or preventing weight gain to overcome the propensity of weight gain associated with the administration of olanzapine or clozapine. Moreover, olanzapine and clozapine are both characterized as non-selective acetylcholine-muscarinic receptor (Ach-M) antagonists. See, e.g., Bymaster, et al., Neuropsychopharmacology (1996) 14(2):87-96; Bymaster, et al., J Clin Psychiatry (1997): 58 Suppl 10:28-36; Zhang and Bymaster, Psychopharmacology (Berl) (1999) 141(3):267-78; Bymaster, et al., Schizophr Res (1999) 37(1):107-22; Sethy, et al., Life Sci. (1996) 58(7):585-90; and Goudie, et al., Behav Pharmacol. (2001) 12(5):303-15. A selective muscarinic receptor M₁ antagonist, for example, telenzepine, is most strongly a M₁R antagonist without any real activity outside of ACh-M. There is no reason to expect that the addition of another M₁R antagonist, e.g., a selective muscarinic receptor M₁ antagonist, for example, telenzepine, would reverse an effect caused by a compound that was already antagonizing this system.

### 2. Methods of Preventing, Inhibiting, Reducing or Reversing Antipsychotic-Induced Weight Gain

a. Conditions Subject to Treatment

The present methods prevent, inhibit, reduce and/or reverse weight gain that is an undesirable side effect of receiving treatment with an antipsychotic such as olanzapine or clozapine by administering to an individual in need thereof a therapeutic amount of one or more selective muscarinic receptor M₁ antagonists.

Olanzapine (tradenames=Zyprexa, Zyprexa Zydis, Zalasta, Zolafren, Olzapin, Rexapin or in combination with fluoxetine as Symbyax) is an atypical antipsychotic. The chemical designation is 2-methyl-4-(4-methyl-1-piperazinyl)-10H-thieno[2,3-b][1,5]benzodiazepine. The molecular formula is C₁₇H₂₀N₄S, which corresponds to a molecular weight of 312.44. Olanzapine is structurally similar to clozapine, and is classified as a thienobenzodiazepine. The chemical structure is:

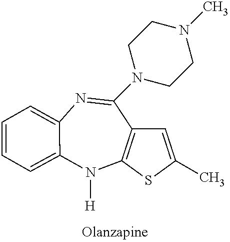

Orally administered olanzapine is indicated for the treatment of schizophrenia, bipolar I disorder (manic or mixed episodes), psychomotor agitation associated with schizophrenia and bipolar I mania, major depressive episodes and treatment resistant depression in adults. Psychological conditions subject to treatment with olanzapine are described in Diagnostic and Statistical Manual of Mental Disorders, Fourth Edition, 2000, American Psychiatric Association, Washington, D.C. (“DSM-IV”).

Clozapine (tradename=Clozaril, Leponex, Fazaclo, Froidir; Denzapine, Zaponex, Klozapol and Clopine) is an antipsychotic medication used in the treatment of schizophrenia. The chemical designation is 8-chloro-11-(4-methyl-1-piperazinyl)-5H-dibenzo(b,e)(1,4)diazepine. The molecular formula is C₁₈H₁₉ClN₄, which corresponds to a molecular weight of 326.823 g/mol. Clozapine is structurally similar to olanzapine and is classified as a dibenzodiazepine. The chemical structure is:

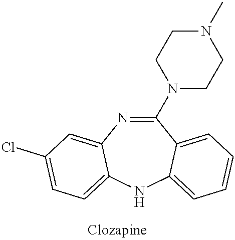

Clozapine is indicated for treatment-resistant schizophrenia. Because of concerns that clozapine may induce potentially fatal agranulocytosis, clozapine is used in patients after other anti-psychotics have failed. Use of clozapine requires weekly blood monitoring for six months followed by monthly testing thereafter.

In the treatment of the indicated psychological disorder, the antipsychotic may be administered as a monotherapy or in combination with another pharmacological agent, for example, lithium, valproate, or an antidepressant, for example, fluoxetine.

The present methods and compositions find use in patients who have been receiving and who are planning to begin receiving administration of an antipsychotic such as olanzapine and clozapine. For example, the methods and compositions find use in patients, e.g., who have been receiving and are continuing to receive administration of the antipsychotic, patients who will be starting another course of treatment with the antipsychotic, and patients who have not yet been treated with the antipsychotic and will be beginning pharmacological treatment with the antipsychotic.

In some embodiments, the patient is to begin or restart a regime of administration of the antipsychotic and/or the patient has not yet experienced weight gain as a result of administration of the antipsychotic. In such cases, co-administration of the antipsychotic with the selective M₁R-antagonist can prevent, inhibit or reduce weight gain caused by administration of the antipsychotic without co-administration of the selective M₁R-antagonist. For example, the patient receiving co-administration of the antipsychotic with the selective M₁R-antagonist can maintain the same weight after undertaking the regime of administration of the antipsychotic as before beginning administration of the antipsychotic, i.e., co-administration of the antipsychotic with the selective M₁R-antagonist can assist with the maintenance of a stable weight. In some cases, the patient experiences reduced weight gain after undertaking the regime of administration of the antipsychotic in combination with the selective M₁R-antagonist, e.g., in comparison to the weight gain experienced in the same patient receiving the antipsychotic without co-administration of the selective M₁R-antagonist. Comparisons to determine the extent of weight gain reduction can also be made with reference to the weight gain experienced by a control patient receiving the antipsychotic without co-administration of the selective M₁R-antagonist, or with reference to the average amount of weight gain of a population of patients receiving the antipsychotic without co-administration of the selective M₁R-antagonist. Comparisons are made with patients receiving a regime of the antipsychotic over the same or a similar period of time.

In some embodiments, the patient is already undergoing and will further continue receiving a regime of administration of the antipsychotic. The patient may or may not have experienced weight gain as a result of administration of the antipsychotic. In patients who have not experienced weight gain, co-administration of the antipsychotic with the selective M₁R-antagonist can prevent or inhibit weight gain that may be caused by administration of the antipsychotic without co-administration of the selective M₁R-antagonist, i.e., co-administration of the antipsychotic with the selective M₁R-antagonist can assist with the maintenance of a stable weight. In patients who have experienced weight gain as a result of administration of the antipsychotic, co-administration of the antipsychotic with the selective M₁R-antagonist can reduce further weight gain or reverse the weight gain caused by administration of the antipsychotic without co-administration of the selective M₁R-antagonist. For example, the patient can experience reduced further weight gain after undertaking the regime of administration of the antipsychotic in combination with the selective M₁R-antagonist, e.g., in comparison to the weight gain experienced in the same patient receiving the antipsychotic without co-administration of the selective M₁R-antagonist over the same or similar time period. In some embodiments, the patient can experience a reversal of the weight gain or a reduction of weight after undertaking the regime of administration of the antipsychotic in combination with the selective M₁R-antagonist.

Depending on the patient, co-administration of the antipsychotic with the selective M₁R-antagonist can result in a 5%, 10%, 15%, 20%, 25%, or greater, reduction of weight gain, e.g., in comparison to the weight gain experienced in the same or a different patient, or the average weight gain of a population of patients, receiving the antipsychotic without co-administration of the selective M₁R-antagonist, over the same or a similar time period.

In some patients, co-administration of the antipsychotic with the selective M₁R-antagonist can result in reversal of antipsychotic-induced weight gain, that is, can effect weight loss. For example, some patients co-administered the antipsychotic with the selective M₁R-antagonist can lose 5%, 10%, 15%, 20%, 25%, 50%, 75% or 100% of the antipsychotic-induced weight gain, e.g., returning to a weight maintained before administration of the antipsychotic without co-administration of the selective KR-antagonist. Some patients co-administered the antipsychotic with the selective M₁R-antagonist can lose weight, for example, losing 3%, 5%, 7%, 10% or more, of their body weight. Some patients co-administered the antipsychotic with the selective M₁R-antagonist can lose 3 lbs., 5 lbs., 10 lbs., 15 lbs., 20 lbs, 25 lbs., or more.

In some embodiments, the patient or subject receiving or intending to receive treatment with olanzapine does not have a movement disorder.

In some embodiments, the patient is receiving olanzapine in combination with lithium and/or valproate.

b. Selective Muscarinic Receptor M₁ Antagonists

Muscarinic antagonists are generally reviewed in Chapter 7 of Goodman and Gilman's The Pharmacological Basis of Therapeutics, 11th Ed., Brunton, Lazo and Parker, Eds., McGraw-Hill (2006), hereby incorporated herein by reference. Exemplified selective muscarinic receptor M₁ antagonists include pirenzepine and telenzepine, the structures of which are shown below.

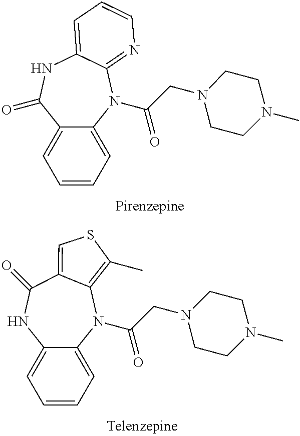

Pirenzepine (5,11-Dihydro-11-[(4-methyl-1-piperazinyl)acetyl]-6H-pyrido[2,3-b][1,4]benzodiazepin-6-one) is manufactured and sold as pirenzepine dihydrochloride by several pharmaceutical companies, including Azupharma (Stuttgart, Germany), Boehringer Ingelheim (Ingelheim, Germany; sold as Gastrozepin®), Dolorgiet (Bonn, Germany). Pirenzepine can be administered in doses from about 50 mg/day to about 200 mg/day, for example, about 100-150 mg/day, or 50, 100, 150, or 200 mg/day. Alternatively, pirenzepine can be administered in doses of about 0.1 mg/kg/day to about 10 mg/kg/day, usually from about 0.7 mg/kg/day to about 5 mg/kg/day. Analogs of pirenzepine also find use in carrying out the present methods. Chemical analogs of pirenzepine are disclosed, for example, in U.S. Pat. Nos. 3,660,380; 3,743,734; and 5,324,832, the disclosures of each of which are hereby incorporated herein by reference in their entirety for all purposes. Further dosage regimens for pirenzepine are disclosed, for example, in U.S. Pat. No. 5,668,155.

Telenzepine (4,9-Dihydro-3-methyl-4-[(4-methyl-1-piperazinyl)acetyl]-10H-thieno[3,4-b][1,5]benzodiazepin-10-one) is commercially available from, for example, Tocris Bioscience (Ellisville, Mo.) and Sigma-Aldrich, Inc. (St. Louis, Mo.) as telenzepine dihydrochloride. Further, the synthesis of telenzepine is disclosed in U.S. Pat. No. 4,381,301, hereby incorporated herein by reference. Telenzepine can be administered in doses from about 0.1 mg per day to about 10 mg per day, for example, about 0.5-5 mg/day, or about 0.1 mg/day, 0.2 mg/day, 0.3 mg/day, 0.4 mg/day, 0.5 mg/day, 1 mg/day, 2 mg/day, 3 mg/day, 4 mg/day, 5 mg/day, 6 mg/day, 7 mg/day, 8 mg/day, 9 mg/day or 10 mg/day. In some embodiments, telenzepine can be administered orally in a dose of from about 0.001 mg/kg to about 0.2 mg/kg, for example, from about 0.005 mg/kg to about 0.1 mg/kg, for example, from about 0.01 mg/kg to about 0.08 mg/kg of body weight, for example in a dose of about 0.001 mg/kg, 0.002 mg/kg, 0.005 mg/kg, 0.008 mg/kg, 0.01 mg/kg, 0.02 mg/kg, 0.03 mg/kg, 0.05 mg/kg, 0.08 mg/kg, 0.1 mg/kg or 0.2 mg/kg. The telenzepine can be administered in one or multiple (e.g., 2, 3, 4, 5 or more) individual administrations, including over an extended period of time, as described herein, in order to achieve the desired results. An individual administration contains the active compound or compounds in an amount of from about 0.001 mg/kg to about 0.2 mg/kg, for example, from about 0.005 mg/kg to about 0.1 mg/kg, for example, from about 0.01 mg/kg to about 0.08 mg/kg of body weight, for example, in an amount of about 0.001 mg/kg, 0.002 mg/kg, 0.005 mg/kg, 0.008 mg/kg, 0.01 mg/kg, 0.02 mg/kg, 0.03 mg/kg, 0.05 mg/kg, 0.08 mg/kg, 0.1 mg/kg or 0.2 mg/kg. Similar dosages are used for parenteral, for example intravenous, treatment. Analogs of telenzepine also find use in carrying out the present methods. Chemical analogs and enantiomers of telenzepine are disclosed, for example, in U.S. Pat. Nos. 3,953,430; 4,168,269; 4,172,831; 4,381,301; 5,140,025 and 5,324,832, the disclosures of each of which are hereby incorporated herein by reference in their entirety for all purposes.

In some embodiments a racemic preparation of telenzepine containing a mixture of (+) and (−) enantiomers is administered. In some embodiments, the (+) or (−) enantiomer of telenzepine is administered. Telenzepine exists in two chirally distinct states separated by an activation barrier of 35.5 kcal/mol (Eveleigh et al., Mol Pharmacol (1989) 35:477-483; and Schudt et al., Eur J Pharmacol (1989) 165:87-96). The (+) form of telenzepine has potent antimuscarinic activity whereas the (−) form is considerably less active. The selectivity of telenzepine appears to vary at different anatomic sites with the (+) form more effective on cortical receptors by a factor of 400 compared to the (−) isomer; on cardiac receptors the selectivity is less and the (+) form is more potent than the (−) form by a factor of 50 (Eveleigh et al., supra). The two forms interconvert slowly and with a half time of approximately 200 hours at 90 degrees (Eveleigh et al., supra). Multiple studies have affirmed that the two forms have distinct activities (Eltze, Eur J Pharmacol (1990) 180:161-168; Eveleigh et al., supra; Feifel et al., Eur J Pharmacol (1991) 195:115-123; Kilian et al., Agents Actions Suppl 34:131-147; Schudt et al., supra).

c. Antidepressants

In some embodiments, the antipsychotic (e.g., olanzapine or clozapine) and selective M₁R-selective antagonist are co-administered with an antidepressant. Antidepressant agents that are not M₁R-selective antagonists for use in the present invention are not limited by their mechanism of action and any class of antidepressant is applicable. For instance, tricyclic antidepressants (TCAs) and analogs thereof, serotonin reuptake inhibitors, monoamine oxidase inhibitors (MAOIs), serotonin agonists and prodrugs thereof, norepinephrine reuptake inhibitors, dopamine reuptake inhibitors, and serotonin reuptake accelerators can all be administered in combination with one or more M₁R-selective antagonists. Serotonin reuptake inhibitors include both selective serotonin reuptake inhibitors (SSRIs) and serotonin-norepinephrine reuptake inhibitors (SNR1s). Norepinephrine reuptake inhibitors include both the specific norepinephrine reuptake inhibitors as well as the mixed norepinephrine-dopamine reuptake inhibitors (NDR1s). Serotonin-norepinephrine-dopamine, or “triple reuptake inhibitors” also find use in the present invention. Other categories of antidepressant can also be used, for example, the tetracyclic antidepressants maprotiline or mianserin, or the agents trazodone, nefazodone, or buspirone; corticotropin releasing factor receptor 1 (CRF1) antagonists, and compounds discovered to have activity in the setting of psychosis or bipolar disorder, including amoxapine, risperidone, quetiapine and aripiprazole.

Tricyclic antidepressants for use in the present invention include amineptine, amitriptyline, clomipramine, desipramine, doxepin, dothiepin, imipramine, nortriptyline, protriptyline, trimipramine, amoxapine and the muscle relaxant cyclobenzaprine. Other unlisted tricyclic antidepressants and analogs thereof can also be used.

In one embodiment, an effective amount of the antipsychotic and the M₁R-selective antagonists is co-administered with an effective amount of a selective serotonin reuptake inhibitor (“SSRI”). Exemplary selective serotonin reuptake inhibitors include citalopram, escitalopram, fluoxetine (racemic or an optical isomer), fluvoxamine, paroxetine and sertraline (and its S-enantiomer, Zoloft®), although SSRIs not listed are applicable. In one embodiment, citalopram (or escitalopram) is co-administered. In one embodiment, an effective amount of fluoxetine (racemic or an optical isomer) is co-administered. In one embodiment, an effective amount of fluvoxamine is co-administered. In one embodiment, an effective amount of sertraline (or its S-enantiomer, Zoloft®) is co-administered. In one embodiment, an effective amount of paroxetine is co-administered. In one embodiment, an effective amount of duloxetine is co-administered.

In one embodiment, an effective amount of one or more serotonin-norepinephrine reuptake inhibitors are co-administered with the antipsychotic and M₁R-selective antagonist. Exemplary serotonin-norepinephrine reuptake inhibitors include milnacipran, mirtazapine, venlafaxine (racemic or an optical isomer), duloxetine, (−)1-(1-dimethylaminomethyl-5-methoxybenzo-cyclobutan-1-yl)cyclohexanol (S33005), DVS-233 (desvenlafaxine), DVS-233 SR and sibutramine, although SNRIs not listed are also of use. Although the mechanism of action of mirtazapine may differ from that of other SNRIs, owing to its apparent dual serotonergic and noradrenergic action, it is considered herein as a member of the SNRI class of antidepressants. In one embodiment, an effective amount of venlafaxine (racemic or an optical isomer) is co-administered. In one embodiment, an effective amount of desvenlafaxine is co-administered. In one embodiment, an effective amount of sibutramine is co-administered. In one embodiment, an effective amount of duloxetine is co-administered. In one embodiment, an effective amount of milnacipran is co-administered. In one embodiment, an effective amount of mirtazapine is co-administered.

In other embodiments, an effective amount of one or more selective norepinephrine reuptake inhibitors is co-administered with the antipsychotic and the M₁R-selective antagonists. Exemplary selective norepinephrine reuptake inhibitors include reboxetine and atomoxetine.

In one embodiment, an effective amount of one or more norepinephrine-dopamine reuptake inhibitors are co-administered with the antipsychotic and the M₁R-selective antagonists. Exemplary norepinephrine-dopamine reuptake inhibitors include amineptine, modafinil, GW353162 and bupropion. In the case of bupropion, metabolites are thought to be responsible for the noradrenergic reuptake blockade. In one embodiment, an effective amount of bupropion is co-administered.

In one embodiment, an effective amount of one or more triple (serotonin-norepinephrine-dopamine) reuptake inhibitors are co-administered with the antipsychotic and the M₁R-selective antagonist. Exemplary triple reuptake inhibitors include indatraline, SEP-225289, DOV 216,303 and (+)-1-(3,4-dichlorophenyl)-3-azabicyclo-[3.1.0]hexane hydrochloride (DOV 21,947).

Monoamine oxidase inhibitors for use in the present invention include befloxatone, brofaromine, deprenyl, isocarboxazid, moclobemide, pargyline, phenelzine, selegiline and tranylcypromine, together with their sustained delivery and transdermal delivery forms.

Additional antidepressants that can be co-administered with the antipsychotic and the M₁R-selective antagonist include maprotiline, tianeptine, nefazodone and trazodone.

Appropriate dosages for antidepressants will depend on the chosen route of administration and formulation of the composition, among other factors. For instance, tricyclic antidepressants are administered at a dose of about 25 to about 600 mg/day, and usually at a dose of about 75 to about 300 mg/day.

Serotonin-reuptake inhibitors are administered at a dose of about 5 to about 400 mg/day, and usually administered at about 20 to about 250 mg/day. In particular, in practicing the present methods, venlafaxine (racemic or an optical isomer) can be administered at about 9 mg to about 225 mg per dose, and is usually administered at about 37.5 mg, 75 mg, 150 mg or 225 mg per dose. Venlafaxine is typically administered at about 25-550 mg/day and usually at about 37.5-375 mg/day, more typically about 75-225 mg/day, and most typically at about 37.5, 75, 150, 225, or 300 mg/day. As appropriate for an individual patient, daily venlafaxine dosages can be divided and administered one time, two times, three times, four or more times a day. Desvenlafaxine can be administered at a dose of about 50-600 mg/day, for example, about 50, 100, 200, 400 or 600 mg/day. Sertraline (or its S-enantiomer, Zoloft®) can be administered in doses ranging from about 50-200 mg/day, usually about 100-150 mg/day. Fluoxetine (racemic or an optical isomer) can be administered in doses ranging from about 5-50 mg/day, usually about 20-40 mg/day. Fluvoxamine can be administered in doses ranging from about 50-300 mg/day, usually about 100-200 mg/day. Paroxetine can be administered in doses ranging from about 10-50 mg/day, usually about 20-40 mg/day.

In carrying out the present methods, citalopram (or escitalopram) can be administered at about 5-60 mg/day, and preferably at about 10, 20 or 30 mg/day. Usually, citalopram is administered once a day, for instance in the morning or in the evening. However, some patients are given dosages of citalopram two or more times a day. Mirtazapine can be administered at a dose of about 5-100 mg/day, for example, about 7.5, 15, 30, 45 or 90 mg/day. Milnacipran can be administered at a dose of about 25-200 mg/day, for example, about 25, 50, 100, 150 or 200 mg/day.

Atypical antidepressants, including bupropion, nefazodone and trazodone are administered at a dose of about 50-600 mg/day, and usually at about 150-400 mg/day. Bupropion can be administered at a dose of about 25-300 mg/day, for example, about 25, 50, 100, 150, 200, 300 mg/day. Monoamine oxidase inhibitors are typically administered at a dose of about 5-90 mg/day, and usually at about 10-60 mg/day.

d. Isomers

All conformational isomers (e.g., cis and trans isomers) and all optical isomers (e.g., enantiomers and diastereomers), racemic, diastereomeric and other mixtures of such isomers, as well as solvates, hydrates, isomorphs, polymorphs and tautomers of the therapeutic agents are within the scope of the present invention.

e. Isotopes

The present invention also includes isotopically-labeled variants of the therapeutic agents, wherein one or more atoms are replaced by one or more atoms having specific atomic mass or mass numbers. Isotopically-labeled variants of the therapeutic agents and prodrugs thereof, as well as isotopically-labeled, pharmaceutically acceptable salts of the therapeutic agents and prodrugs thereof, are within the scope of the present invention. In certain circumstances substitution with heavier isotopes, such as deuterium (²H), can provide increased metabolic stability, which offers therapeutic advantages such as increased in vivo half-life or reduced dosage requirements. Isotopically-labeled variants of the therapeutic agents of this invention and prodrugs thereof can generally be prepared according to methods known to those skilled in the art by substituting an isotopically-labeled reagent for a non-isotopically labeled reagent.

f. Administration

i. Duration of Administration

Usually, the one or more M₁R-selective antagonists are administered to the individual co-extensive with the time period of administration of the antipsychotic, for example, olanzapine or clozapine. However, in some cases, administration of the one or more M₁R-selective antagonists can begin before the beginning of treatment with the antipsychotic and/or can continue after the discontinuing treatment with antipsychotic. This can be over an extended period of time. For example, the methods can be carried out for at least 20 days, in some embodiments for at least 40, 60, 80 or 100 days, and in some embodiments for at least 150, 200, 250, 300, 350 days, 1 year or longer. In some embodiments, the methods are carried out for the rest of the life of the patient. Certain individuals receive the present treatment methods for longer than a year, for example, at least 400, 450, 500, 550, 600, 650, 700, 800, 900, 1000 days. However, individuals can be successfully treated with the present methods for 2 years, 3 years, 4 years or longer. Administration of the M₁R-selective antagonists can be continuous or discontinuous (i.e., intermittent) over the time period of treatment.

ii. Scheduling

Generally, in practicing the present methods, therapeutically effective amounts of one or more M₁R-selective antagonists are co-administered with a therapeutically effective amount of the antipsychotic. The co-administered pharmacological agents can be administered together or separately, simultaneously or at different times. When administered, the M₁R-selective antagonists and the antipsychotic independently can be administered once, twice, three, four times daily or more or less often, as needed. Preferably, the administered pharmacological agents are administered once daily. Preferably, the administered active agents are administered at the same time or times, for instance as an admixture. One or more of the pharmacological agents can be administered in a sustained-release formulation.

For certain patients, the methods are carried out concurrently administering the one or more M₁R-selective antagonists and the antipsychotic from the initiation of treatment. For certain patients, the methods are carried out by first administering the one or more M₁R-selective antagonists, and then subsequently co-administering the antipsychotic, or vice versa. In some embodiments, the patient initially can be given the one or more M₁R-selective antagonists alone for as long as 3 days, 5 days, 7 days, 10 days, 14 days, 20 days, or 30 days before commencing administration of the antipsychotic. In some embodiments, the patient initially can be given the antipsychotic alone for as long as 3 days, 5 days, 7 days, 10 days, 14 days, 20 days, 30 days, or longer, before commencing administration of the one or more M₁R-selective antagonists.

When administered for the purpose of facilitating weight loss or suppressing appetite, the one or more M₁R-selective antagonists, alone or in combination, can be administered prophylactically to prevent undesirable weight gain or maintain a stable weight, or therapeutically to achieve a desired weight loss and maintain such weight loss for a sustained period of time.

iii. Routes of Administration

As such, administration of one or more M₁R-selective antagonists, alone or in combination with the antipsychotic can be achieved in various ways, including oral, buccal, parenteral, including intravenous, intradermal, subcutaneous, intramuscular, transdermal, transmucosal, intranasal, etc., administration. The one or more M₁R-selective antagonists can be administered by the same or different route of administration when co-administered with the antipsychotic.

In some embodiments, one or more M₁R-selective antagonists, alone or in combination, can be administered in a local rather than systemic manner, for example, in a depot or sustained release formulation.

iv. Methods of Determining Appropriate Dosages

Administered dosages for M₁R-selective antagonists and the antipsychotic (e.g., olanzapine or clozapine) are in accordance with dosages and scheduling regimens practiced by those of skill in the art. General guidance for appropriate dosages of all pharmacological agents used in the present methods is provided in Goodman and Gilman's The Pharmacological Basis of Therapeutics, 11th Edition, 2006, supra, and in a Physicians' Desk Reference (PDR), for example, in the 59^{th} (2005) or 60^{th} (2006) Eds., Thomson PDR, each of which is hereby incorporated herein by reference. Published dosages for M₁R-selective antagonists are for indications distinct from treatments to treat obesity or to promote weight loss or inhibit weight gain. In the compositions and methods of the present invention, efficacious dosages of M₁R-selective antagonists and the antipsychotic for practicing the present invention can be equal to or less than (e.g., about 25, 50, 75 or 100%) the dosages published for other indications.

The appropriate dosage of M₁R-selective antagonists and the antipsychotic will vary according to several factors, including the chosen route of administration, the formulation of the composition, patient response, the severity of the condition, the subject's weight, and the judgment of the prescribing physician. The dosage can be increased or decreased over time, as required by an individual patient. Usually, a patient initially is given a low dose, which is then increased to an efficacious dosage tolerable to the patient.

Determination of an effective amount is well within the capability of those skilled in the art, especially in light of the detailed disclosure provided herein. In some embodiments, an efficacious or effective amount of a combination of one or more M₁R-selective antagonists and the antipsychotic is determined by first administering a low dose or small amount of an M₁R-selective antagonist alone, and then incrementally increasing the administered dose or dosages, adding the antipsychotic as needed, until a desired effect of is observed in the treated subject with minimal or no toxic side effects. In some embodiments, an efficacious or effective amount of a combination of one or more M₁R-selective antagonists and the antipsychotic is determined by first administering a low dose or small amount of the antipsychotic alone, and then incrementally increasing the administered dose or dosages, adding one or more M₁R-selective antagonists as needed, until a desired effect of is observed in the treated subject with minimal or no toxic side effects. Applicable methods for determining an appropriate dose and dosing schedule for administration of a combination of the present invention are described, for example, in Goodman and Gilman's The Pharmacological Basis of Therapeutics, 11th Edition, 2006, supra; in a Physicians' Desk Reference (PDR), supra; in Remington: The Science and Practice of Pharmacy, 21^{st} Ed., 2005, supra; and in Martindale: The Complete Drug Reference, Sweetman, 2005, London: Pharmaceutical Press., and in Martindale, Martindale: The Extra Pharmacopoeia, 31st Edition., 1996, Amer Pharmaceutical Assn, each of which are hereby incorporated herein by reference.

Dosage amount and interval can be adjusted individually to provide plasma levels of the active compounds which are sufficient to maintain therapeutic effect. Preferably, therapeutically effective serum levels will be achieved by administering single daily doses, but efficacious multiple daily dose schedules are included in the invention. In cases of local administration or selective uptake, the effective local concentration of the drug may not be related to plasma concentration. One having skill in the art will be able to optimize therapeutically effective local dosages without undue experimentation.

Applicable quantitative doses for pirenzepine and telenzepine are described above. With respect to exemplified quantitative dosing of olanzapine, initial doses of 5 to 10 mg are administered, with a target dose of 10 mg/day within several days. Efficacy is demonstrated in a dose range of 10 to 15 mg/day. Olanzapine is generally not indicated for use in doses above 20 mg/day.

With respect to exemplified quantitative dosing of clozapine, initial doses of about 12.5 mg are administered once or twice daily and then increased with daily dosage increments of 25-50 mg/day, if well tolerated, to achieve a target dose of 300-450 mg/day by the end of 2 weeks. Subsequent dosage increments are made about once or twice weekly, in increments of about 100 mg. Daily dosing can continue on a divided basis as an effective and tolerable dose level is sought. While many patients may respond adequately at doses between 300-600 mg/day, it may be necessary to raise the dose to the 600-900 mg/day range to obtain an acceptable response.

### 3. Compositions

The present invention further provides pharmaceutical compositions comprising a mixture of a therapeutically effective amount of one or more M₁R-selective antagonists and the antipsychotic (e.g., olanzapine or clozapine). In some embodiments, the M₁R-selective antagonists are selected from the group consisting of telenzepine, pirenzepine and mixtures thereof.

A combination of one or more M₁R-selective antagonists and the antipsychotic can be administered to a subject, e.g., a human patient, a domestic animal such as a cat or a dog, independently or together in the form of their pharmaceutically acceptable salts, or in the form of a pharmaceutical composition where the compounds are mixed with suitable carriers or excipient(s) in a therapeutically effective amount, e.g., at doses effective to effect desired weight loss or maintenance or prevent undesired weight gain.

A combination of one or more M₁R-selective antagonists and the antipsychotic of this invention can be incorporated into a variety of formulations for therapeutic administration. More particularly, a combination of the present invention can be formulated into pharmaceutical compositions, together or separately, by formulation with appropriate pharmaceutically acceptable carriers or diluents, and can be formulated into preparations in solid, semi-solid, liquid or gaseous forms, such as tablets, capsules, pills, powders, granules, dragees, gels, slurries, ointments, solutions, suppositories, injections, inhalants and aerosols.

Suitable formulations for use in the present invention are found in, for example, in Remington: The Science and Practice of Pharmacy, 21^{st} Ed., 2005, supra; Martindale: The Complete Drug Reference, Sweetman, 2005, London: Pharmaceutical Press.; Niazi, Handbook of Pharmaceutical Manufacturing Formulations, 2004, CRC Press; and Gibson, Pharmaceutical Preformulation and Formulation: A Practical Guide from Candidate Drug Selection to Commercial Dosage Form, 2001, Interpharm Press, which are hereby incorporated herein by reference. The pharmaceutical compositions described herein can be manufactured in a manner that is known to those of skill in the art, i.e., by means of conventional mixing, dissolving, granulating, dragee-making, levigating, emulsifying, encapsulating, entrapping or lyophilizing processes. The following methods and excipients are merely exemplary and are in no way limiting.

In one embodiment, a combination of one or more M₁R-selective antagonists and the antipsychotic (e.g., olanzapine or clozapine) is prepared for delivery in a sustained-release, controlled release, extended-release, timed-release or delayed-release formulation, for example, in semi-permeable matrices of solid hydrophobic polymers containing the therapeutic agent. Various types of sustained-release materials have been established and are well known by those skilled in the art. Current extended-release formulations include film-coated tablets, multiparticulate or pellet systems, matrix technologies using hydrophilic or lipophilic materials and wax-based tablets with pore-forming excipients (see, for example, Huang, et al. Drug Dev. Ind. Pharm. 29:79 (2003); Pearnchob, et al. Drug Dev. Ind. Pharm. 29:925 (2003); Maggi, et al. Eur. J. Pharm. Biopharm. 55:99 (2003); Khanvilkar, et al., Drug Dev. Ind. Pharm. 228:601 (2002); and Schmidt, et al., Int. J. Pharm. 216:9 (2001)). Sustained-release delivery systems can, depending on their design, release the compounds over the course of hours or days, for instance, over 4, 6, 8, 10, 12, 16, 20, 24 hours or more. Usually, sustained release formulations can be prepared using naturally-occurring or synthetic polymers, for instance, polymeric vinyl pyrrolidones, such as polyvinyl pyrrolidone (PVP); carboxyvinyl hydrophilic polymers; hydrophobic and/or hydrophilic hydrocolloids, such as methylcellulose, ethylcellulose, hydroxypropylcellulose, and hydroxypropylmethylcellulose; and carboxypolymethylene.

The sustained or extended-release formulations can also be prepared using natural ingredients, such as minerals, including titanium dioxide, silicon dioxide, zinc oxide, and clay (see, U.S. Pat. No. 6,638,521, herein incorporated by reference). Exemplified extended release formulations that can be used in delivering a combination of one or more M₁R-selective antagonists and the antipsychotic (e.g., olanzapine or clozapine) of the present invention include those described in U.S. Pat. Nos. 6,635,680; 6,624,200; 6,613,361; 6,613,358, 6,596,308; 6,589,563; 6,562,375; 6,548,084; 6,541,020; 6,537,579; 6,528,080 and 6,524,621, each of which is hereby incorporated herein by reference. Controlled release formulations of particular interest include those described in U.S. Pat. Nos. 6,607,751; 6,599,529; 6,569,463; 6,565,883; 6,482,440; 6,403,597; 6,319,919; 6,150,354; 6,080,736; 5,672,356; 5,472,704; 5,445,829; 5,312,817 and 5,296,483, each of which is hereby incorporated herein by reference. Those skilled in the art will readily recognize other applicable sustained release formulations.

For oral administration, a combination of one or more M₁R-selective antagonists and the antipsychotic can be formulated readily by combining with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as a cross-linked polyvinyl pyrrolidone, agar, or alginic acid or a salt thereof such as sodium alginate.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

The compounds can be formulated for parenteral administration by injection, e.g., by bolus injection or continuous infusion. For injection, a combination of one or more M₁R-selective antagonists and the antipsychotic can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Preferably, a combination of the invention can be formulated in aqueous solutions, preferably in physiologically compatible buffers such as Hanks's solution, Ringer's solution, or physiological saline buffer. Formulations for injection can be presented in unit dosage form, e.g., in ampules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

Systemic administration can also be by transmucosal or transdermal means. For transmucosal or transdermal administration, penetrants appropriate to the barrier to be permeated are used in the formulation. For topical administration, the agents are formulated into ointments, creams, salves, powders and gels. In one embodiment, the transdermal delivery agent can be DMSO. Transdermal delivery systems can include, e.g., patches. For transmucosal administration, penetrants appropriate to the barrier to be permeated are used in the formulation. Such penetrants are generally known in the art. Exemplified transdermal delivery formulations that can find use in the present invention include those described in U.S. Pat. Nos. 6,589,549; 6,544,548; 6,517,864; 6,512,010; 6,465,006; 6,379,696; 6,312,717 and 6,310,177, each of which are hereby incorporated herein by reference.

For buccal administration, the compositions can take the form of tablets or lozenges formulated in a conventional manner.

In addition to the formulations described previously, a combination of one or more M₁R-selective antagonists and the antipsychotic of the present invention can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also can comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

### 4. Kits

The pharmaceutical compositions of the present invention can be provided in a kit. In certain embodiments, a kit of the present invention comprises one or more M₁R-selective antagonists and the antipsychotic (e.g., olanzapine or clozapine) in separate formulations. In certain embodiments, the kits comprise one or more M₁R-selective antagonists and the antipsychotic (e.g., olanzapine or clozapine) within the same formulation. In certain embodiments, the kits provide at least one of the one or more M₁R-selective antagonists and the antipsychotic independently in uniform dosage formulations throughout the course of treatment. In certain embodiments, the kits provide at least one of the one or more M₁R-selective antagonists and the antipsychotic independently in graduated dosages over the course of treatment, either increasing or decreasing, but usually increasing to an efficacious dosage level, according to the requirements of an individual.

In one embodiment, the kits comprise one or more pharmaceutical compositions comprising one or more M₁R-selective antagonists selected from the group consisting of telenzepine and pirenzepine.

In one embodiment, the kits comprise one or more pharmaceutical compositions comprising olanzapine. In one embodiment, the kits comprise one or more pharmaceutical compositions comprising clozapine.

Further embodiments of the compositions contained in the kits are as described herein.

### EXAMPLES

The following examples are offered to illustrate, but not to limit the claimed invention.

### Example 1

### Telenzepine Inhibits Olanzapine-Induced Weight Gain

Forty (40) Wistar-Han female rats (n=10/group) were implanted with Alzet minipumps in order to determine the effectiveness of Telenzepine (TZP) in preventing weight gain associated with Olanzapine (OLZ) administration. The rats were divided into the following four groups:

Group 1—Control: Vehicle (NMP:DMSO:PEG) alone

Group 2—OLZ (10 mg/kg/day) alone

Group 3—TZP (5 mg/kg/day) alone

Group 4—OLZ (10 mg/kg/day)+TZP (5 mg/kg/day)

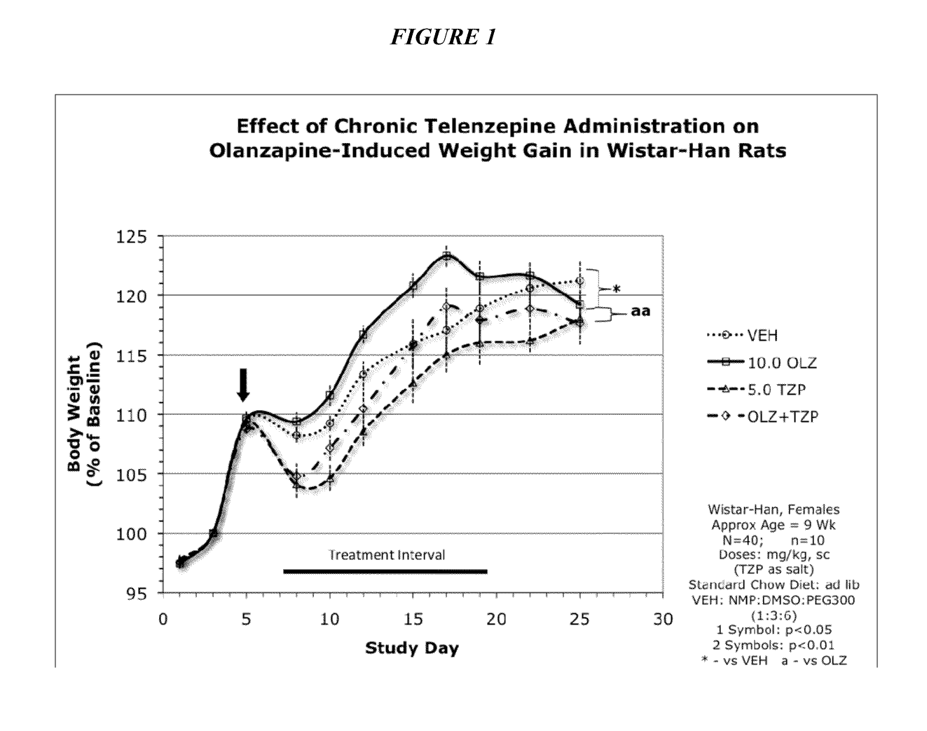

The results are shown in FIG. 1.

The co-administration of telenzepine with olanzapine reversed olanzapine-induced weight gain in a rat model. Two 2-way repeated measures ANOVAs were run: 1) Comparing No Treatment (VEH) to OLZ alone (addressing the question of whether or not OLZ induced weight gain), 2) Comparing OLZ alone to OLZ+TZP (addressing the question of whether or not TZP co-administration prevented OLZ-Induced weight gain).

With respect to the ANOVA for 1), VEH vs OLZ, significant main effects were returned for Time (p<0.0001) & Treatment (p=0.042; indicated by * on FIG. 1: Solid Line vs Dotted Line), as well as a significant Time×Treatment Interaction (p=0.026). Therefore, rats gained significant amounts of weight over the duration of treatment, regardless of the drug condition. Additionally, rats receiving OLZ alone gained significantly more weight than those receiving No Treatment. Further, the size of the effect for OLZ-Induced weight gain was differential over the treatment interval (with the greatest separation evident on Treatment Day 10).

With respect to the ANOVA for 2), OLZ vs OLZ+TZP, Significant main effects were returned for Time (p<0.0001) & Treatment (p=0.01; indicated by “aa” on FIG. 1: Solid line vs Dot-Dash-Dot Line), however the Time×Treatment Interaction resulted in a p value just falling short of the level of significance (p=0.075). Therefore, rats gained significant amounts of weight over the duration of treatment, regardless of the drug condition. Additionally, rats receiving OLZ alone gained significantly more weight than those receiving OLZ+TZP. However, the magnitude of this difference was not altered significantly by treatment duration.

In both analyses, the follow-up pair-wise comparisons for each treatment day failed to reach the level of significance after using the Bonferroni adjusted alpha, due to the vast number of comparisons. The significant main effects for Treatment returned by the 2-way ANOVAs are sufficient to conclude that: 1) OLZ induced a significant amount of weight gain over No Treatment controls, and 2) Co-administration of TZP resulted in a significant reduction in OLZ-induced weight gain. Assignment of significant differences to specific treatment days is not possible under this statistically rigorous approach.

In summary, peak weight gain induced by OLZ was observed on Treatment Day 12, with a difference of 6.2% gain between the groups. At this point, co-administration of TZP resulted in a weight gain that was similar to Vehicle treated rats, resulting in a non-significant 2% difference between OLZ+TZP and Vehicle treated rats (therefore, the difference from OLZ alone was 4.2% at this point).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of preventing, inhibiting, reducing or reversing weight gain in a subject receiving treatment with an antipsychotic selected from olanzapine and clozapine, comprising co-administering to the subject therapeutically effective amounts of the antipsychotic and an M₁R-selective antagonist.

2. The method of claim 1, wherein the antipsychotic and the M₁R-selective antagonist are concurrently administered.

3. The method of claim 1, wherein the antipsychotic and the M₁R-selective antagonist are sequentially administered.

4. The method of claim 1, wherein the subject is human.

5. The method of claim 1, wherein delivery of at least one of the antipsychotic and the M₁R-selective antagonist is sustained release.

6. The method of claim 1, wherein the M₁R-selective antagonist is selected from the group consisting of telenzepine and pirenzepine.

7. The method of claim 1, wherein the M₁R-selective antagonist is telenzepine.

8. The method of claim 1, wherein the antipsychotic is olanzapine.

9. The method of claim 1, wherein the antipsychotic is clozapine.

10. A composition comprising a mixture of therapeutically effective amounts of an antipsychotic selected from olanzapine and clozapine and an M₁R-selective antagonist.

11. The composition of claim 10, wherein the mixture is formulated for sustained release delivery of at least one of the antipsychotic and the M₁R-selective antagonist.

12. The composition of claim 10, wherein the M₁R-selective antagonist is selected from the group consisting of telenzepine and pirenzepine.

13. The composition of claim 10, wherein the M₁R-selective antagonist is telenzepine.

14. The composition of claim 10, wherein the antipsychotic is olanzapine.

15. The composition of claim 10, wherein the antipsychotic is clozapine.

16. A kit comprising a combination of therapeutically effective amounts of an antipsychotic selected from olanzapine and clozapine and an M₁R-selective antagonist.

17. The kit of claim 16, wherein the antipsychotic and the M₁R-selective antagonist are formulated in a mixture.

18. The kit of claim 16, wherein at least one of the antipsychotic and the M₁R-selective antagonist are formulated for sustained release delivery.

19. The kit of claim 16, wherein the M₁R-selective antagonist is selected from the group consisting of telenzepine and pirenzepine.

20. The kit of claim 16, wherein the M₁R-selective antagonist is telenzepine.

21. The kit of claim 16, wherein the antipsychotic is olanzapine.

22. The kit of claim 16, wherein the antipsychotic is clozapine.
